(19) United States
(12) Patent Application Publication
(10) Pub. No.: US 2024/0115855 A1
(43) Pub. Date: Apr. 11, 2024

(54) System For Management Of Musculoskeletal Disorders

(71) Applicants: Mary Reaston, Carlsbad, CA (US); Phil Reaston, Carlsbad, CA (US)

(72) Inventors: Mary Reaston, Carlsbad, CA (US); Phil Reaston, Carlsbad, CA (US)

(21) Appl. No.: 17/961,245

(22) Filed: Oct. 6, 2022

(51) Int. Cl. A61N 1/08; A61B 5/00; A61N 1/32; G16H 10/60; G16H 15/00; G16H 40/67; G16H 50/20

(52) U.S. Cl. CPC A61N 1/08; A61B 5/00; A61N 1/32; G16H 10/60; G16H 15/00; G16H 40/67; G16H 50/20; A61B 5/0031; A61B 5/0205

(57) ABSTRACT

A system for management of musculoskeletal disorders (SMMD) that is designed to integrate physiological monitoring and functional assessment, i.e., muscles, nerves, brain waves, cardiac, blood flow, heat rate, range of motion, global positioning, temperature and functional abilities into a single integrated test unit. The SMMD is operated by proprietary software which utilizes wireless sensor(s) that are interchangeable, rechargeable and that can process data without the need to be connected by a cable to a computer and/or wireless device such as a smartphone. The SMMD is also capable of offering preventative assessment as well as site-specific treatment. The SMMD comprises: at least one wireless sensor, at least one prevention and evaluation device, a wireless processing circuit, a receiving and transmitting interface, and a data receiving unit produces and output that indicates a subject's test results.

### TECHNICAL FIELD

The invention generally pertains to medical evaluation systems, and more particularly to a system for management of musculoskeletal disorders that provides preventative and evaluative functions for injuries and disorders that affect a human body's movement or musculoskeletal system such as muscles, tendons, ligaments, nerves, discs and blood vessels.

### BACKGROUND ART

It is well known that muscle compensation patterns can be used to differentiate chronic versus acute injuries. Acute injuries are typically exemplified by muscle spasm and hyperactivity. Chronic injuries are typically associated with compensation, bilateral changes, and absence of a reflection/relaxation response. Many physicians deal with complaints and injuries that involve musculoskeletal disorders (MSDs), particularly cervical, thoracic, lumbosacral and extremities. All muscle groups of the body, from the cervical including the mid, thoracic, lumbosacral and upper and lower extremities, can be monitored. This is extremely beneficial because MSD may be chronic or acute, with no physical indicators. Direct palpitation of a MSD can be very limiting, thus rarely can this method reveal the nature or type of injury. Another limited method of diagnosis relies on static testing and on individual tests that are performed separately, which are less reliable than combining and integrating evaluations, as well as limiting in that multiple areas cannot be tested. To enhance and correlate tests, physiological parameters, cardiac response, brain wave activity and blood flow can also be monitored.

The combination of tests does not rely on the coefficients of variance but measures muscle and other body activity while simultaneously monitoring additional parameters either alone or in combination. This dynamic integration provides an analysis of Type I versus Type II motor units with other body functions, which coordinate with an effort produced by an individual who is being tested. Furthermore, when disc pathology is present there is a chemical released in the individual's blood supply, which causes ischemic changes in the surrounding musculature. The benefit of quantifying these conditions in a non-invasive and portable manner is that the amount of ischemia/vasoconstriction in the surrounding blood tissue correlates with the degree of disc pathology, thereby allowing preventive or evaluative recommendations to be made, i.e. surgery versus physical therapy as well as less reliance on opioids with better diagnosis.

In the past, various technological tests such as EMG, range of motion, cardiac information or functional assessments were relied upon to determine MSDs. However, when the tests are performed alone they are less objective. By integrating all of the functions it is possible to acquire a more objective assessment.

There is difficulty in preventing and evaluating MSDs, especially with OSHA's ergonomic standards. With the implementation of the Americans with Disabilities Act (ADA) MSDs have increased. MSDs are also often misdiagnosed, for example referred pain from the scalene muscle can mimic the symptoms of neuropathy, carpal tunnel syndrome or even thoracic outlet syndrome. The misdiagnosed problems are in reality a muscle problem in the front of the neck, or muscle problems can also refer to pain in the back and be misdiagnosed as disc pathology which might result in unnecessary surgery.

Individuals who have undergone MSD treatment often receive no relief and show no improvement. The lack of improvement can be attributed to the inability to accurately identify the MSD. Furthermore, individuals who have undergone back surgery resulted in failed back syndrome or have no relief of symptomology. This can also be attributed to an inability to properly diagnose and age a MSD. In the past, prior art has experienced difficulty performing a proper diagnosis.

Previously, it was not possible to incorporate all of functions required to perform a comprehensive diagnostic test in a reliable, cost effective and user-friendly manner. The uniqueness of the functionality is the utilization of remote and wireless sensor(s) which provide portability and increased ease of use. The wireless sensor(s) can have a disposable component, and any proprietary sensor(s) that are not disposable have their own battery-recharging unit. This allows for no down-time during monitoring and, since the sensor(s) can be interchangeable, if one sensor(s/as) is not functioning, a replacement is immediately available.

MSDs are a leading cause of lost work, time and productivity. MSDs result in lost man-hours and increased medical costs. In fact, MSDs are responsible for many Workers Compensation claims. MSDs are often non-specific, difficult to diagnose and treat, and current subjective diagnostics can be a “process of elimination”. In addition, it is often difficult to determine when a MSD has occurred. A search of the prior art did not disclose literature or patents that mad directly on the claims of the instant invention. However, the following U.S. patents are considered related:

| PATENT NO. | INVENTOR | ISSUED |
|---|---|---|
| 10,750,977 | Kording, et al | Aug. 25, 2020 |
| 11,069,439 | Heismann, et al | Jul. 20, 2021 |
| 11,211,161 | Lyman, et al | Dec. 28, 2021 |

The U.S. Pat. No. 10,750,977 patent discloses a medical evaluation system including an I/O module, a processing module, and an analysis module. The I/O module receives sensory data obtained by a motion sensor in a mobile device carried by a patient in a non-clinical environment. The processing module extracts data from the sensory data received from the sensor in the mobile device. The analysis module device surrogate biomarkers from the relevant data. The surrogate biomarkers represent a state or a progression of a medical condition of the patient. The mobile device may be a mobile phone and the sensor may include an accelerometer or a gyroscope.

The U.S. Pat. No. 11,069,439 patent discloses a method for controlling an evaluation system for medical images of a patient. Medical images acquired at different time points are stored in an image database. Upon reception of a user command associated with a time-dependent evaluation command including at least one evaluation parameter describing an evaluation to be performed and identification information. Medical images fulfilling a relevancy criterion depending on the evaluation parameter are retrieved from the database. The images are registered to each other and a composite evaluation image data set is derived from the images.

The U.S. Pat. No. 11,211,161 patent discloses a medical scan interface feature evaluator system that generates an ordered image-to-prompt mapping by selecting a set of user interface features to be displayed. A set of medical scans and the ordered mapping are transmitted to a set of client devices. A set of responses are generated by each client device in response to sequentially displaying a set of medical scans in conjunction with a mapped user interface feature. Response score data is generated by comparing each response to truth annotation data of the corresponding medical scan. Interface feature score data corresponding to each user interface feature is generated and is used to generate a ranking of user interface features.

For background purposes and as indicative of the art to which the invention relates, reference may be made to the following remaining patents found in the search.

| PATENT NO. | INVENTOR | ISSUED |
|---|---|---|
| 4,586,515 | Berger | May 6, 1986 |
| 4,667,513 | Moss | May 26, 1987 |
| 4,688,581 | Konno | Aug. 25, 1987 |
| 4,742,832 | Kauffmann, et al | May 10, 1988 |
| 4,800,897 | Nilsson | Jan. 31, 1989 |
| 4,805,636 | Barry, et al | Feb. 21, 1989 |
| 4,834,057 | McLeod, Jr. | May 30, 1989 |
| 4,845,987 | Kenneth | Jul. 18, 1989 |
| 4,886,073 | Dillon, et al | Dec. 12, 1989 |
| 4,928,709 | Allison, et al | May 29, 1990 |
| 4,938,476 | Brunelle, et al | Jul. 3, 1990 |
| 5,012,820 | Meyer | May 7, 1991 |
| 5,038,795 | Roush, et al | Aug. 13, 1991 |
| 5,042,505 | Mayer, et al | Aug. 27, 1991 |
| 5,056,530 | Butler, et al | Oct. 15, 1991 |
| 5,462,065 | Cusimano, et al | Oct. 31, 1995 |
| 5,513,651 | Cusimano, et al | May 7, 1996 |
| 6,678,549 | Cusimano, et al | Jan. 13, 2004 |
| 8,535,224 | Reaston, et al | Sep. 17, 2013 |
| 8,568,312 | Reaston, et al | Oct. 29, 2013 |
| 2002/0091308 | Kipshidze, et al | Jul. 11, 2002 |
| 2002/0198473 | Kumar, et al | Dec. 26, 2002 |
| 2003/0083720 | Peterson, et al | May 1, 2003 |
| 2003/0135129 | Cusimano, et al | Jul. 17, 2003 |
| 2006/0058699 | Vitiello, et al | Mar. 16, 2006 |
| 2007/0230270 | Calhoun | Oct. 4, 2007 |
| 2009/0281448 | Wright, et al | Nov. 12, 2009 |

Jovanove et al “A Wireless Body Area Network of Intelligent Motion Sensors for Computer Assisted Physical Rehabilitation” Journal of NeuroEngineering and Rehabilitation 2005, 2:6 dol:10,1186/1743-0003-2-6, Published Mar. 1, 2005, cited by examiner.

### DISCLOSURE OF THE INVENTION

The system for management of musculoskeletal disorders (SMMD) is designed to integrate physiological monitoring and functional assessment, i.e., muscles, nerves, brain waves, cardiac, blood flow, heat rate, range of motion, temperature and functional abilities into a single integrated test unit. The SMMD is operated by proprietary software which utilizes wireless sensor(s) that are interchangeable, rechargeable and that can process data without the need to be connected by a cable to a computer and/or smartphone. The SMMD also capable of offering preventative assessment as well as site-specific treatment.

The SMMD comprises: at least one wireless sensor, at least one prevention and evaluation device having an input and an output, a wireless processing circuit that receives and process data transmitted from the at least one sensor and the output from the at least one prevention and evaluation device, a receiving and transmitting interface that receives output from the wireless processing circuit and produces an output, and a data receiving unit that receives and processes the output from the receiving and transmitting interface, and produces and output that indicates a subject's test results.

The wireless sensors 26 can be selected from, but not limited to: at least one electromyography (EMG) sensor, at least one electrocardiogram (EKG) sensor, at least one electroencephalograph (EEG) sensor, at least one galvanic skin response (GRS) sensor, at least one range of motion (ROM) sensor, at least one functional capacity evaluation (FCE) sensor, at least one blood flow sensor, at least one temperature sensor, at least one level sensor, at least one pinch and grip sensor, and at least one global positioning satellite (GPS) sensor. The at least one prevention and evaluation device can include at least one treatment delivery device, at least one massage device, at least one ultrasound device, at least one heat device, and at least one electrical stimulation device. A power source that can be battery power, mains utility power or solar power provides power to the CPU and converts voltage to a regulated d-c voltage.

The CPU can be a computer such as a desktop computer, a laptop computer, a tablet computer, a smartphone, or a smartwatch. The software, the CPU/computer operates is either conventionally, coded computer software or an app. In addition to prevention and evaluation, the SMMD can also delivery and sensor verification, data transfer in real time comprising file transfer protocol (FTP), electronic media, remote control, and cloud storage.

In view of the above disclosure, the primary object of the invention is to provide a system for management of musculoskeletal disorders (MDSD), which includes almost all muscle groups in a human body. The system offers preventative and evaluative functions, as well as delivering site-specific treatment based on the diagnostic evaluations.

In addition to the primary object of the invention, it is also an object of the invention to provide a system for management of musculoskeletal disorders that:
is easy to use,
can be used on humans or animals,
does not have a high learning curve,
is essentially maintenance free,
can perform multiple evaluations simultaneously and transfer data in real-time, or store data,
is effective for establishing base-line test results for insurance purposes,
can improve a person's quality of life by disclosing the evaluative test results and providing injury prevention assessment, and
is cost effective from both a manufacturer's and subject's point of view.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is an elevational view of a system for management of musculoskeletal disorders with one wireless sensor and one prevention and evaluation device.

FIG. 2 is an elevational view of the system for management of musculoskeletal disorders, with multiple wireless sensors and multiple prevention and evaluation devices.

### BEST MODE FOR CARRYING OUT THE INVENTION

The best mode for carrying out the invention is presented in terms that disclose a preferred embodiment of a system for management of musculoskeletal disorders (SMMD). Many medical issues that people experience are the result of musculoskeletal disorders (MSDS) or injuries, which affect a human body's movement or musculoskeletal system such as muscles, tendons, ligaments, nerves, discs and blood vessels. Common MSDs include carpal tunnel syndrome, tendonitis, muscle/tendon strain or ligament sprain.

There have been efforts to provide a means of evaluating preventing and/or treating MSDs. But most are not completely effective, require complicated medical procedures, and are time consuming. As a result, it is evident that there is a need for a device or system that could accurately evaluate a person for a MSD in quick and efficient manner, and even offer preventative assessment and/or site-specific treatment of MSD.

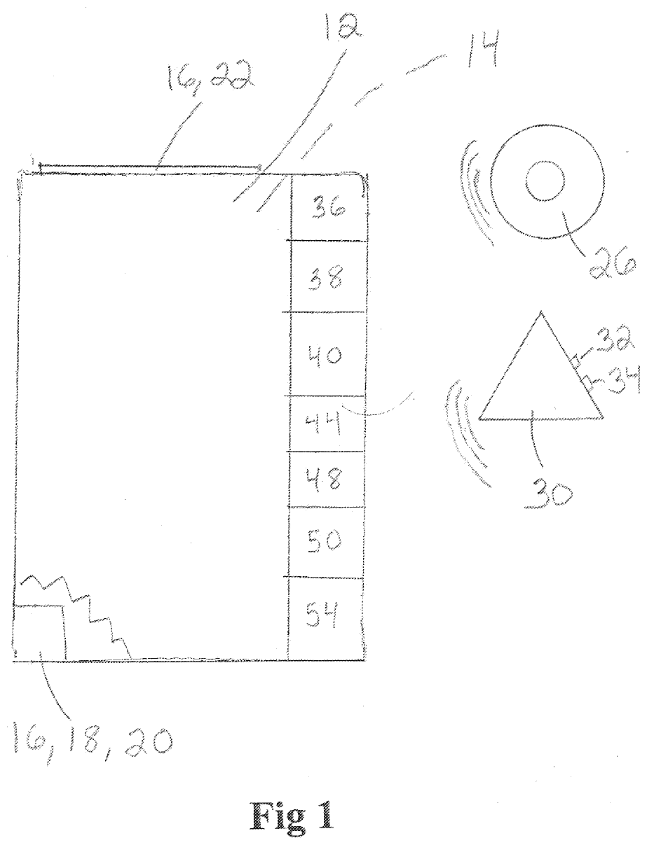

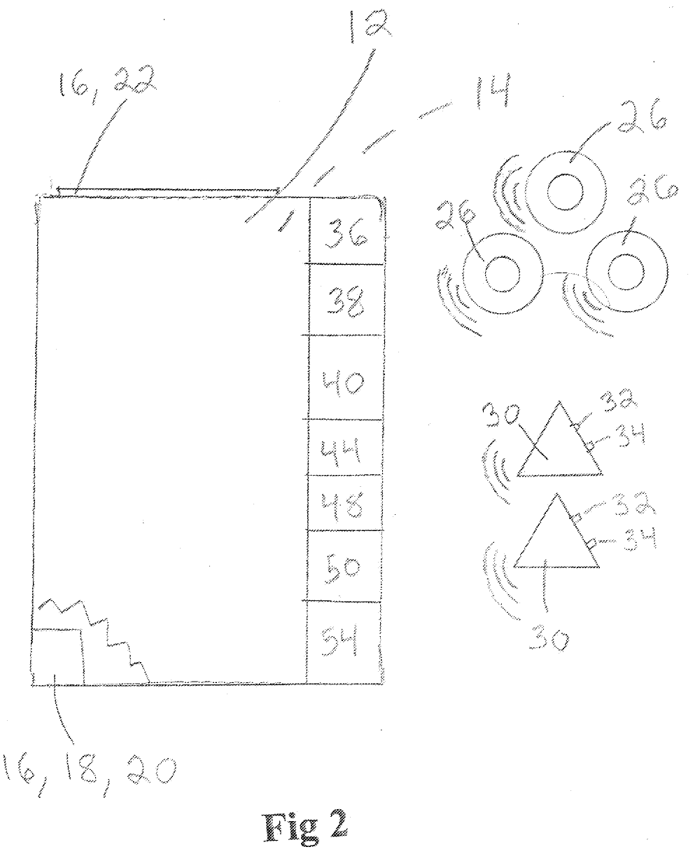

The SMMD 10, as shown in FIGS. 1 and 2, is such a system. The SMMD 10 is comprised of a central processing unit (CPU) 12, that is configured to operate software 14. At least one, but preferably, multiple wireless sensors 26 are attached to a selected area of a subject who is undergoing a testing protocol, and transmit data acquired from the testing protocol, and at least one prevention and evaluation device 30 having an input 32 and an output 34, with the input 32 receiving data from a subject who is under-going an evaluation protocol. A plurality of wireless processing circuits 36 each comprise a signal conditioner 38 that receives and processes the transmitted data from the wireless sensors 26 and the prevention and evaluation devices 30, and provides an output.

A wireless processor 40 receives and processes the output from the signal conditioner 38, and produces an output, a receiving interface 44 receives the output from the wireless processing circuit 36, and produces an output. If desired or required, a transmitting interface can be used in combination with the receiving interface 44. Optionally, a wireless control circuit 48 can receive the output from the receiving interface (and transmitting interface if utilized), and produce and output. Note: the wireless control circuit is optional because it is not a required component of the SMMD 10. A data acquisition circuit 50 receives and processes the output from the transmitting interface (and/or the wireless control circuit), and produces an output. And finally, a data controller/processor 54 receives the output from the data acquisition circuit 50. The data controller/processor 54 operates in combination with the software 14 in the CPU 12 to provide an output that is indicative of a subject's test results. A power source 16 provides the required voltage/current to the CPU 12. The power source 16 is comprised of either battery power 18, mains utility power 20 or solar power 22, and is able to convert the voltage to a regulated d-c voltage.

The wireless sensors 26 can be selected from, but not limited to: at least one electromyography (EMG) sensor, at least one electrocardiogram (EKG) sensor, at least one electroencephalograph (EEG) sensor, at least one galvanic skin response (GRS) sensor, at least one range of motion (ROM) sensor, at least one functional capacity evaluation (FCE) sensor, at least one blood flow sensor, at least one temperature sensor, at least one level sensor, at least one pinch and grip sensor, and at least one global positioning satellite (GPS) sensor.

The prevention and evaluation devices 30 are selected from, but not limited to: at least one treatment delivery device, at least one massage device, at least one ultrasound device, at least one heat device, and at least one electrical stimulation device. While the prevention and evaluation devices have inputs and outputs, they also have the capability of transmitting data wirelessly to the CPU 12.

The wireless sensor(s) 26, the prevention and evaluation devices 30, and the receiving and transmitting interface 44 utilize a wireless system that can include: WiFi, Bluetooth, Zigbee, Z-Wave, GSM, UMTS. LTE, WLAN, 802.IIag, IoT, Radar, Satellite and Waveguide. Additionally, the CPU 12 can be a computer and can have complete wireless functionality, using any of the above wireless systems. The type of computer the CPU comprises can include a desktop computer, a laptop computer, a tablet computer, a smartphone, or even a smartwatch. Also, the software 14 can be configured as conventional computer software or as an app.

The CPU 12 also includes additional functionality, including sensor verification, treatment delivery, and data transfer comprising file transfer protocol (FIP), electronic media, remote control and cloud storage. It should be noted that while the SMMD 10 is preferably designed as a wireless device, wired connections could also be utilized. Cables could be used to connect the sensors and prevention and evaluation devices, as well as any other peripheral devices.

While the invention has been described in detail and pictorially shown in the accompanying drawings, it is not to be limited to such details, since many changes and modifications may be made to the invention without departing from the spirit and the scope thereof. Hence, it is described to cover any and all modifications and forms which may come within the language and scope of the claims.

1. A system for management of musculoskeletal disorders (MSDs) comprising:
a central processing unit (CPU) configured to operate software,
at least one wireless sensor, at least one prevention and evaluation device comprising an input and an output
a wireless processing circuit configured to receive and process data transmitted from the at least one sensor and the output from the at least one prevention and evaluation device,
a receiving interface configured to receive output from the wireless processing circuit and produce an output, and
a data receiving unit configured to receive and process the output from the receiving interface, and produce and output that indicates a subject's test results.

2. The system for management of musculoskeletal disorders of claim 1, wherein the at least one wireless sensor is selected from the group consisting of:
at least one electromyography (EMG) sensor,
at least one electrocardiogram (EKG) sensor,
at least one electroencephalograph (EEG) sensor,
at least one galvanic skin response (GRS) sensor,
at least one range of motion (ROM) sensor,
at least one functional capacity evaluation (FCE) sensor,
at least one blood flow sensor,
at least one temperature sensor,
at least one level sensor,
at least one pinch and grip sensor, and
at least one global positioning satellite (GPS) sensor.

3. The system for management of musculoskeletal disorders of claim 1, wherein the prevention and evaluation device are selected from the group consisting of:
at least one treatment delivery device,
at least one massage device,
at least one ultrasound device,
at least one heat device, and
at least one electrical stimulation device.

4. The system for management of musculoskeletal disorders of claim 1, wherein the CPU comprises a power source selected from the group consisting of battery power, mains utility power, a battery, and solar power.

5. The system for management of musculoskeletal disorders of claim 4, wherein the battery is a rechargeable battery.

6. The system for management of musculoskeletal disorders of claim 1, wherein the wireless processing circuit comprises:
a signal conditioner that receives, filters and amplifies the outputs from the at least one wireless sensor and at least one prevention and evaluation device, and produces an output, and
a wireless processor that receives and processes output from the signal conditioner and produces an output.

7. The system for management of musculoskeletal disorders of claim 1, wherein the at least one wireless sensor and the receiving and transmitting interface utilizes a wireless system that is selected from the group consisting of WiFi, Bluetooth, Zigbee, Z-Wave, GSM, UMTS, LTE, WLAN, 802.IIag, IoT, Radar, Satellite and Waveguide.

8. The system for management of musculoskeletal disorders of claim 1, wherein the data receiving unit comprises a data controller/processor that receives and processes the output from the wireless processor, the data controller/processor operating in combination with the software to produce an output that indicates a subject test results.

9. The system for management of musculoskeletal disorders of claim 1, wherein the CPU is located in a computer comprising wireless capability.

10. The system for management of musculoskeletal disorders of claim 1, further comprising a transmitting interface that functions in combination with the receiving interface.

11. A system for management of musculoskeletal disorders comprising a central processing unit (CPU) configured to operate software:
a plurality of wireless sensors that are attached to a selected area of a subject that is undergoing a testing protocol, and are configured to transmit data acquired from the testing protocol,
a plurality of prevention and evaluation devices, each device comprising an input and an output, with the input configured to receive data from a subject that is undergoing an evaluation devices protocol, and
a plurality of wireless processing circuits each comprising:
a signal conditioner configured to receive and process the transmitted data from the wireless sensors and the prevention and evaluation devices and produce an output, a wireless processor configured to receive and process the output from the signal conditioner and produce an output,
a receiving interface configured to receive the output from the wireless processor and produce an output,
a data acquisition circuit configured to receive and process the output from the receiving interface and produce an output,
a data controller/processor configured to receive the output from the data acquisition circuit, the data controller/processor operating in combination with the software to produce an output that is indicative of a subject's test results,
treatment delivery sensors for delivering site-specific treatment that is selected from the group consisting of ultrasound, electrical stimulation, heat, cooling, massage, and global positioning.

12. The system for management of musculoskeletal disorders of claim 11, wherein the plurality of wireless sensors is selected from the group consisting of:
EMG sensors,
EKG sensors,
EEG sensors,
temperature sensors,
level sensors,
heart rate sensors,
blood flow sensors,
oxygen sensors,
range of motion (ROM) sensors,
functional capacity evaluation (FCE) sensors,
pinch and grip sensors, and
global positioning satellite sensors.

13. The system for management of musculoskeletal disorders of claim 11, wherein the prevention and evaluation devices are selected from the group consisting of:
massage devices,
ultrasound devices,
heat devices,
electrical stimulation devices, and
treatment delivery devices.

14. The system for management of musculoskeletal disorders of claim 11, wherein the subject comprises a human or an animal.

15. The system for management of musculoskeletal disorders of claim 11, wherein the wireless sensors and the receiving and transmitting interface utilizes a wireless system that is selected from the group consisting of WiFi, Bluetooth, Zigbee, Z-Wave, GSM, UMTS, LTE, WLAN, 802.IIag, IoT, Radar, Satellite and Waveguide.

16. The system for management of musculoskeletal disorders of claim 11, wherein the power source comprises at least one of either a battery, utility power source or solar power that converts the voltage to a regulated direct current voltage.

17. The system for management of musculoskeletal disorders of claim 11, wherein the CPU located in a computer having wireless capability.

18. The system for management of musculoskeletal disorders of claim 11, wherein the CPU further comprises functionality selected from the group consisting of:
sensor verification,
treatment delivery, and
data transfer;
file transfer protocol (FTP),
electronic media,
remote control, and
cloud storage.

19. The system for management of musculoskeletal disorders of claim 11, further comprising means for aging, diagnosis and treatment an injury.

20. The system for management of musculoskeletal disorders of claim 11, further comprising real time transmission of data to diagnose and treat a person, or to store the data.

21. The system for management of musculoskeletal disorders of claim 11, wherein the CPU is located in a computer with software configured as an app.
